(19) United States
(12) Patent Application Publication
(10) Pub. No.: US 2025/0352536 A1
(43) Pub. Date: Nov. 20, 2025

(54) NEURORESTORATION COMPOSITIONS IMPLEMENTING MULTIPLE NEUROPLASTICITY INDUCING MECHANISMS OF ACTION

(71) Applicant: Neuro-Innovators, Inc., Sewickley, PA (US)

(72) Inventors: John Howison Schroeder, Sewickley, PA (US); Peter C. Doyle, Sherborn, MA (US); Ian Jeffery Reynolds, Leefdaal (BE); Christopher J. Doyle, West Lebanon, NH (US); Florenta Aura Kullmann, Pittsburgh, PA (US); Mark Andrew Cochran, Ruckersville, VA (US)

(21) Appl. No.: 19/222,410

(22) Filed: May 29, 2025

Related U.S. Application Data
(63) Continuation of application No. PCT/US23/81509, filed on Nov. 29, 2023.
(60) Provisional application No. 63/428,722, filed on Nov. 29, 2022.

(51) Int. Cl. A61K 31/4709; A61K 31/155; A61K 31/381; A61K 31/4184; A61K 45/06

(52) U.S. Cl. CPC A61K 31/4709; A61K 31/155; A61K 31/381; A61K 31/4184; A61K 45/06

(57) ABSTRACT

One embodiment provides a neurorestoration compound or composition implementing multiple neuroplasticity inducing mechanisms of action including a mixture of pharmacologically effective amounts of Cilostazol and Metformin and Telmisartan. A second embodiment provides a neurorestoration compound or composition implementing multiple neuroplasticity inducing mechanisms of action including a mixture of pharmacologically effective amounts of Cilostazol and Metformin and Duloxetine. The neurorestoration compound implementing multiple neuroplasticity inducing mechanisms of action is believed to be particularly well suited for stroke rehabilitation.

### RELATED APPLICATIONS

The present application is a continuation of International Patent Application PCT/US2023/081509 filed Nov. 29, 2023 and published Jun. 6, 2024 as Publication WO 2024/118705 which application and publication are incorporated herein by reference.

International Patent Application PCT/US2023/081509 claims the benefit of U.S. provisional patent application Ser. No. 63/428,722 filed Nov. 29, 2022 titled “Neurorehabilitation Compound Implementing Multiple Neuroplasticity Inducing Mechanisms of Action” which application is incorporated herein by reference.

### BACKGROUND OF THE INVENTION

### 1. Field of the Invention

This invention generally relates to neurorehabilitation implementing multiple neuroplasticity inducing mechanisms of action for enhancing neuro-restoration or the building of new neurons, axons, and synapses and remyelination in the human brain.

### 2. Background Information

As reported in the May 1, 2019 journal Neurology out-of-pocket costs are rapidly on the rise for commonly prescribed neurologic medications. This is of great significance as it has been estimated that one in six people lives with a neurologic disease or disorder. It has further been estimated in 2019 that the annual cost of treating neurologic disorders in the United States is more than $500 billion (with the current cost expected to be substantially higher).

Aside from cost to patients there are extraneous stresses on the economy from brain disorders and diseases. It is estimated that brain disorders and diseases cost U.S. economy over $2 trillion annually with these costs underscoring the scale of the need and opportunity for greater research and innovative new treatments to improve health and drive prosperity. See for earlier estimates Information Technology & Innovation Foundation Jul. 11, 2016.

Unfortunately, the field of commercial pharmacology and drug development is, generally, focused on the development of single molecules addressing a single mechanism of action to provide comprehensive therapeutics to cure a disease. This strategy has often proven to be a failure, particularly in the central nervous system (CNS) diseases or conditions.

Success of a pharmacological agent for neurologic use is limited, among other issues, by its capacity to cross the blood brain barrier (BBB) in sufficient amount to produce the necessary effect. An effective, potentially efficacious agent is often rendered clinically ineffective due the inability to pass in a therapeutically sufficient quantity across the blood brain barrier, and crossing this barrier becomes the governing factor of an agent's clinical and commercial success.

A combinatorial pharmacological composition is defined herein as the combination of two or more distinct therapeutic agents into a single composition. The PHARNEXT™ brand agent [PO #304u10] has shown us the potential synergistic therapeutic benefits of three drugs delivered simultaneously to cure or slow the progression of a genetic disorder known as Charcot Marie Tooth (technically it is a group of inherited disorders which cause nerve damage mostly in arms and legs. Weakness in limbs, hammer toes, and loss of sensation in limbs are the common symptoms). There are other examples of new FDA therapeutics with Food and Drug Administration (FDA) approval using two drugs or molecules concurrently to form a combinatorial pharmacological composition, but the use of combination therapies to treat central nervous system (CNS) diseases and its consequences remains rare. Currax Pharmaceuticals' CONTRAVE® brand drug is naltrexone HCL and bupropion HCL extended release was approved in 2014 by the FDA and has become a popular obesity medication. The CONTRAVE® brand drug works in two parts of the brain to help some adults control their eating, resulting in sustained weight loss.

The term Neurorestoration encompasses multiple processes in the brain. Following an injury to the brain, which may be acute or progressive, the cellular components in the brain are damaged. The consequence of the damage is an impairment of function of the injured individual. Neurorestoration includes the repair or replacement of the damaged cellular components of the brain, which can be termed neurogenesis or gliogenesis or remyelination. Neurorestoration also includes the reconnection of physiological circuits within the brain, which can be termed neuroplasticity. Neurorestoration is facilitated by the removal or cessation of damaging processes, such as apoptotic cell death or inhibition of brain metabolism. Neurorestoration may also include re-establishing blood flow to injured parts of the brain, which can be termed revascularization, and a resolution of over-activated inflammatory responses. Neuro-restoration thus incorporates a number of distinct processes that contribute to the repair of the brain after injury. For a further discussion of neurorestoration, see Azad T D, Veeravagu A, Steinberg G K. Neurorestoration after stroke. Neurosurg Focus. 2016 May; 40(5): E2 MOVE EARLIE to 10.

Combinatorial pharmacotherapy (also known as polypharmacology) is defined herein as a pharmacotherapeutic protocol implementing at least one combinatorial pharmacological composition comprised of at least two drugs.

A compound is defined herein as a combination of drugs having a common delivery and dosage mechanism e.g., drugs combined within the same pill, liquid, aerosol etc. This compound may also be referred to as a Combination Drug Product or CDP herein.

There remains a great need for enhancing neuro-restoration in the human brain by an effective pharmacotherapeutic protocol.

### SUMMARY OF THE INVENTION

One embodiment of the present invention provides a neuro-restoration CDP, compound or composition implementing multiple neurorehabilitation mechanisms of action including at least one of Cilostazol and Metformin and at least one of Telmisartan and Duloxetine.

The term composition herein follows the U.S. Patent Law definition of composition of matter, namely, the combination of two or more substances, whether combined by chemical union or mechanical mixture and whether they be gases, fluids, powders or solids. Compounds are chemical substances made up of two or more elements that are chemically bound together in a fixed ratio, and mixtures of chemical substances made up of two or more elements that are chemically bound together in a fixed ratio.

One embodiment of the present invention provides a neurorehabilitation CDP, compound or composition implementing multiple neurorestoration mechanisms of action including Cilostazol and Metformin and Telmisartan.

One embodiment of the present invention provides a neurorehabilitation compound, CDP or composition implementing multiple neurorestoration mechanisms of action including Cilostazol and Metformin and Duloxetine.

The present invention provides a combinatorial pharmacotherapy via the neurorestoration compound or CDP which provides for enhancing neuro-restoration, by implementing a combinatorial pharmacological composition combining two or more pharmacologic molecules in a pharmacotherapeutic protocol wherein the two or more pharmacologic molecules concurrently moderates/manages the function of two or more mechanisms of action necessary for enabling the creation of neurons, axons, synapses, and or remyelination, improving neuroplasticity, improving blood flow and resolving of inflammation to enhance the environment for and or accelerate the growth of neuronal networks damaged by or malfunctioning as a result of disease or trauma.

### DESCRIPTION OF THE PREFERRED EMBODIMENTS

The various embodiments and examples of the present invention as presented herein are understood to be illustrative of the present invention and not restrictive thereof and are non-limiting with respect to the scope of the invention.

The present invention focuses on the restoration of neuro-function damaged or lost as a result of disease or trauma. The present invention provides a combinatorial pharmacological composition which combines two or more of the 160+ molecules known to enhance neurorestoration, including the creation of neurons, axons, synapses, and or remyelination, neuroplasticity, revascularization, resolution of inflammation and removing damaging processes to restore brain function, specifically two of Cilostazol, Metformin, Telmisartan and Duloxetine, wherein each of the molecules of the combinatorial pharmacological composition targets several different mechanisms of action and each of which effects a mechanism of action differently.

Each combinatorial pharmacological composition or compound of the present invention may include a vasodilator to improve blood flow to the brain and/or an agent to increase flow of active ingredients across the blood brain barrier.

### Neuroplasticity

Neuroplasticity describes the composite changes in connectivity in human brains in response to stimulations, exercises, and experiences, such as learning to play an instrument or recovering the use of one's arm with therapy after a stroke. There is a long history in the published literature of doctors observing such changes, but its recognition was largely anecdotal, based on individual observations. Recent advances have enabled detection and measurement of neuronal changes and growth in animal brains, providing us with the ability to observe and understand neuroplasticity in humans. New technologies that are based on functional imaging of the human brain allow the direct measurement of connectivity between brain regions in the living human brain. These technologies also allow the determination of impaired connectivity following brain injury, and potentially the restoration of connectivity (which is the definition of neuroplasticity) following therapeutic interventions. There are numerous published studies showing tangible positive changes in post-stroke patients (referred to as chronic stroke patients) and others with degenerative brain challenges, in response to various treatment regimes.

Neurorestoration effectively creates a pliable substrate within which rehabilitation can act to promote neuroplasticity, allowing individual neurons, synapses, and whole neural networks to experience enhanced and efficient reconfiguration. Neurorestoration should ideally take advantage of this plasticity by exercising, stimulating, and enhancing configurations that are beneficial and therapeutic to the patient. Because of this, targeted stimuli and rehabilitative tasks that exercise valuable functions as well as broad general regions of neural territory are a logical choice.

The term “neuroplasticity” in the context of this application is meant to cover all interpretations of plasticity, or modifiability, in the brain, such as: Neurogenesis, or the creation of new neurons; Gliogenesis, or the creation of new glia, Apoptosis of specific cells, or synapse removal, the selective elimination of neuronal connections, which is a normal part of neural re-wiring; Synaptogenesis, or the creation of new, or enhancement of existing (but not yet signaling) synapses between neurons, including branching of neural or axonal arbors; Synaptic plasticity, namely changes in the communication strength of synapses, either increasing in strength, decreasing, becoming more or less inhibited, or any other beneficial change or modulation of synapses; Changes induced by interactions with, or other changes in, other non-neural cells in the brain, e.g., astrocytes, oligodendrocytes, microglia, pericytes or other cells derived from the blood or vasculature; Changes in genetic expression, e.g., changes in expression of genes that affect brain activity, including but not limited to changes in cell receptors, neurotransmitters, or cell-signaling pathways or pathway controlling brain metabolism; and or Changes at the interface of native human tissue and implanted medical devices.

### Neurorestoration Compound or Composition

The various embodiments and examples of the present invention as presented herein are understood to be illustrative of the present invention and not restrictive thereof and are non-limiting with respect to the scope of the invention.

There is accepted knowledge regarding various neurorestorative physiologic processes (also known as mechanisms of action) with the human brain that either promote or suppress neuro-restoration. The present invention targets the attenuation of neurorestorative physiologic processes individually and concurrently. The present invention categorizes seven key neurorestorative physiologic processes of which two are Inhibitory and five excitatory. The Inhibitory include: 1) Apoptosis 2) Neuro-inflammation; The Excitatory include: 3) Neurogenesis 4) Synaptogenesis 5) Mitochondrial protection and Bioenergetics 6) Remyelination 7) Angiogenesis.

Apoptosis is a form of programmed cell death that occurs in multicellular organisms. Biochemical events lead to characteristic cell changes (morphology) and death. These changes most generally include blebbing, cell shrinkage, nuclear fragmentation, chromatin condensation, DNA fragmentation, and mRNA decay. For reference, the average adult human loses between 50 and 70 billion cells each day due to apoptosis. For an average human child between eight and fourteen years old, approximately twenty to thirty billion cells die per day. In contrast to necrosis, which is a form of traumatic cell death that results from acute cellular injury, apoptosis is a highly regulated and controlled process that confers advantages during an organism's life cycle.

Unlike necrosis, apoptosis produces cell fragments called apoptotic bodies that phagocytes are able to engulf and remove before the contents of the cell can spill out onto surrounding cells and cause damage to them. Apoptosis can be initiated through one of two pathways. In the intrinsic pathway the cell kills itself because it senses cell stress, while in the extrinsic pathway the cell kills itself because of signals from other cells. Weak external signals may also activate the intrinsic pathway of apoptosis. Both pathways induce cell death by activating caspases, which are proteases, or enzymes that degrade proteins. The two pathways both activate initiator caspases, which then activate executioner caspases, which then kill the cell by degrading proteins indiscriminately. In addition to its importance as a biological phenomenon, defective apoptotic processes have been implicated in a wide variety of diseases. Excessive apoptosis causes atrophy, whereas an insufficient amount results in uncontrolled cell proliferation, such as cancer. Some factors like Fas receptors and caspases promote apoptosis, while some members of the Bcl-2 family of proteins inhibit apoptosis.

Neuroinflammation is inflammation of the nervous tissue. It may be initiated in response to a variety of cues, including infection, traumatic brain injury, toxic metabolites, or autoimmunity. In the central nervous system (CNS), including the brain and spinal cord, microglia are the resident innate immune cells that are activated in response to these cues. The CNS is typically an immunologically privileged site because peripheral immune cells are generally blocked by the blood brain barrier (BBB), a specialized structure composed of astrocytes and endothelial cells. However, circulating peripheral immune cells may surpass a compromised BBB and encounter neurons and glial cells expressing major histocompatibility complex molecules, perpetuating the immune response. Although the response is initiated to protect the central nervous system from the infectious agent, the effect may be toxic and widespread inflammation as well as further migration of leukocytes through the blood brain barrier. Neuroinflammation is implicated in contributing to a variety of chronic neurologic and somatic illnesses including Alzheimer's disease (AD), Parkinson's disease (PD), and depression. Neuroinflammation is also a prominent response to acute neurologic injuries such as stroke and traumatic brain injury.

Neurogenesis is the process by which new neurons are produced from the differentiation of neural stem cells (NSCs). It occurs in all species of animals (except the porifera (sponges) and placozoans). Types of NSCs include neuroepithelial cells (NECs), radial glial cells (RGCs), basal progenitors (BPs), intermediate neuronal precursors (INPs), subventricular zone astrocytes, and subgranular zone radial astrocytes, among others. Neurogenesis is most active during embryonic development and is responsible for producing all the various types of neurons of the organism, but it continues throughout adult life in a variety of organisms. Once born, neurons do not divide (e.g., mitosis), and many will live the lifespan of the animal. Neurogenesis may effectively be defined as the formation of new neurons from neural stem and progenitor cells which occurs in various brain regions such as the subgranular zone of dentate gyrus in the hippocampus and the subventricular zone of lateral ventricles. The integration of new neurons into brain circuits may contribute to neurorestoration. New glial cells are produced in the brain by a process termed gliogenesis. Gliogenesis is also a consequence of the differentiation of stem cells that are resident in the brain. The production of new glial cells from brain stem cells can contribute to the repair of connectivity in the brain. For example, oligodendrocyte precursor cells can differentiate into mature oligodendrocytes which can then re-form the myelin sheath surrounding neurons and thus improve neuronal function.

Within the brain, mitochondria serve as the primary producers of ATP to meet the high energy requirements of individual neurons. Through its electron transport chain (ETC), mitochondria generate most of this ATP in an oxygen-dependent manner, with toxic reactive oxidative species (ROS) also released from the same process. Over time an accumulation of this ROS can severely damage the mitochondrial population within the neuron, ultimately causing apoptosis of the affected neurons. Mitochondrial dysfunction is often implicated in disorders of the brain, in particular Parkinson's disease (PD), an incurable movement disorder caused by the progressive central nervous system (CNS) degeneration of dopaminergic neurons (DA). Compared to other neurons, DA neurons are more vulnerable to ROS due to their intrinsic pace making ability. As a consequence, these neurons are under constant oxidative stress that can cause irreparable damage to mitochondria. Mitochondrial function is regulated by biochemical control mechanisms and by alteration in the production of proteins that contribute to mitochondrial energy production. Some drugs improve mitochondrial function by altering the activity or expression of these proteins. Mitochondrial or bioenergetic protection the action or mechanism of promoting energy production by the mitochondria while limiting ROS production.

Remyelination is the process of propagating oligodendrocyte precursor cells to form oligodendrocytes to create new myelin sheaths on demyelinated axons in the central nervous system. Myelin formation is an essential part of neuronal development, because neurons wrapped with myelin are much more efficient in transmitting electrical signals. This is a process naturally regulated in the body and tends to be very efficient in a healthy central nervous system. The formation of myelin is a plastic process and can change in response to neuronal activity associated with repetitive motor tasks or cognitive functions. Damage to or loss of the myelin sheath is associated with serious neurological diseases including multiple sclerosis and adrenoleukodystrophy. Damage to the myelin sheath can be repaired by oligodendrocyte precursor cells migrating to a lesion, differentiating into oligodendrocytes and re-wrapping the axon. The process creates a thinner myelin sheath than normal, but it helps to protect the axon from further damage, from overall degeneration, and proves to increase conductance once again. The processes underlying remyelination are under investigation in the hope of finding treatments for demyelinating diseases, such as multiple sclerosis.

Angiogenesis continues the growth of the vasculature by processes of sprouting and splitting. Vasculogenesis is the embryonic formation of endothelial cells from mesoderm cell precursors, and from neovascularization, although discussions are not always precise (especially in older texts). The first vessels in the developing embryo form through vasculogenesis, after which angiogenesis is responsible for most, if not all, blood vessel growth during development and in disease. Angiogenesis is a normal and vital process in growth and development, as well as in wound healing and in the formation of granulation tissue. Regenerating nerve axons and neural stem cells require access to damaged brain areas that angiogenesis provides. However, it is also a fundamental step in the transition of tumors from a benign state to a malignant one, leading to the use of angiogenesis inhibitors in the treatment of cancer.

One preferred embodiment of the present invention provides a neurorestoration compound or composition which engages multiple neurorestoration mechanisms of action and includes pharmacologically effective amounts of Cilostazol and Metformin and Telmisartan. A second preferred embodiment of the present invention provides a neurorestoration compound implementing multiple neurorestoration mechanisms of action and includes pharmacologically effective amounts of Cilostazol and Metformin and Duloxetine. The preferred embodiments of the present invention restore neurofunction damaged or lost as a result of disease or trauma. The preferred embodiments of the present invention create an optimal environment within which neurorestoration can happen, allowing individual neurons, synapses, and whole neural networks to experience enhanced and efficient reconfiguration. Neurorestoration promoted by the use of combinatorial pharmacotherapy should ideally take advantage benefits provided by exercising, stimulating, and enhancing configurations that are beneficial and therapeutic to the patient. Because of this, targeted stimuli and rehabilitative tasks that exercise valuable functions as well as associated axonal tracts are a logical choice.

As noted above the present invention provides a combinatorial pharmacological composition or composition or CDP which combines two or more of the 160+ molecules known to enhance neuro-restoration (i.e., the creation of neurons, axons, synapses, and or myelin sheaths to restore brain function) and implemented within a combinatorial pharmacotherapy. The combinatorial pharmacotherapy developed under this invention may be in combination with a structured, clinically supervised (or unsupervised for certain activities) rehabilitation protocol (for example but not limited to: physical therapy, VR/AR Gaming therapy, exercise, or repetitive daily activity). The combinatorial pharmacotherapy developed under this invention may be in combination with different forms of central and/or peripheral nervous system stimulation (for example but not limited to brain stimulation including non-invasive and invasive forms, magnetic or electrical, such as transcranial magnetic stimulation, transcranial electrical stimulation, deep brain stimulation through implanted neuroelectrodes, spinal cord stimulation, or vagus nerve stimulation). Necessary daily activities can provide sufficient repetition to promote the rebuilding of the necessary neuronal, axonal, synaptic, and or myelin pathways to restore/enhance function in the presence of an enhanced neurologic environment for the creation of new neurons, axons, synapsis, and or myelin sheaths.

### Cilostazol

Cilostazol has a formula C₂₀H₂₇N₅O₂ and is a selective inhibitor of phosphodiesterase, which in turn increases the activation of intracellular CAMP and thereby inhibits platelet aggregation. An increase in cAMP results in an increase in the active form of protein kinase A (PKA), which is directly related with an inhibition in platelet aggregation. Preclinical studies have shown that cilostazol can decrease microglial activation and thus reduce inflammation, decrease apoptosis, and prevent loss of oligodendrocytes. Cilostazol may also promote neurogenesis and revascularization. Clinical studies have shown effects of cilostazol on inflammation and blood vessel formation.

Cilostazol has been noted as a powerful alternative to aspirin in certain aspects. In previous clinical trials for example, cilostazol has been found to significantly reduce the incidence of recurrent stroke, with fewer hemorrhagic events, compared with aspirin. See Huang Y, Cheng Y, Wu J, Li Y, Xu E, Hong Z, et al; Cilostazol versus Aspirin for Secondary Ischaemic Stroke Prevention Cooperation Investigators. Cilostazol as an alternative to aspirin after ischaemic stroke: a randomised, double-blind, pilot study. Lancet Neurol. 2008; 7:494-499. See also Nakamura T, Tsuruta S, Uchiyama S. Cilostazol combined with aspirin prevents early neurological deterioration in patients with acute ischemic stroke: a pilot study. J Neurol Sci. 2012; 313:22-26.

Cilostazol may have several delivery methods or routes of administration, but oral or IV delivery is preferred in the compound of the invention. Effective amounts of Cilostazol in the daily dosage of the compound of the present invention is less than 200 mg, more preferably less than 100 mg, and most preferably less than 50 mg. The effective amount of Cilostazol in the compound of the present invention may be less than 25 mg. Effective amounts of Cilostazol in the daily dosage is 1 mg to 200 mg in the compound of the present invention, preferably 10 mg to 100 mg.

### Metformin

Metformin has a formula C₄H₁₁N₅ and is well established as a main first-line medication for the treatment of type 2 diabetes, particularly in people who are overweight. It is also used in the treatment of polycystic ovary syndrome (PCOS). Metformin is generally regarded as safe and well-tolerated. Metformin may have multiple mechanisms of action, but a primary effect may involve the activation of AMP-activated protein kinase (AMPK). Metformin may also partially inhibit complex I of the mitochondrial electron transport chain.

Metformin is a biguanide drug that reduces blood glucose levels by decreasing glucose production in the liver, decreasing intestinal absorption, and increasing insulin sensitivity. Metformin decreases both basal and postprandial blood glucose levels. In PCOS, Metformin decreases insulin levels, which then decreases luteinizing hormone and androgen levels. This effect of metformin results in the improvement of mitochondrial function and metabolism. Metformin also decreases inflammation and inhibits apoptosis. Additional preclinical studies show that metformin promotes remyelination and increases neuroplasticity. Clinically, metformin has been associated with improved outcomes following stroke.

Metformin, like Cilostazol, may have several delivery methods or routes of administration, but oral or IV delivery is preferred. Effective amounts of Metformin in the daily dose of the compound of the present invention are less than 2000 mg per day, more preferably less than 850 mg, more preferably less than 500 mg, and most preferably less than 250 mg per dose. Metformin may be less than 125 mg per dose of the compound of the present invention. The effective amounts of Metformin in the daily dosage are 30 mg to 2000 mg in the compound of the present invention, preferably 40 mg to 850 mg, more preferably 50 mg to 600 mg and 500 mg being an efficient effective amount.

### Telmisartan

Telmisartan has a chemical formula C₃₃H₃₀N₄O₂ is an angiotensin II receptor blocker that shows high affinity for the angiotensin II receptor type 1 (AT₁), with a binding affinity 3000 times greater for AT₁ than AT₂. In addition to blocking the renin-angiotensin system, Telmisartan acts as a selective activator of peroxisome proliferator-activated receptor gamma (PPAR-γ), a central regulator of insulin and glucose metabolism. Telmisartan's dual mode of action may provide protective benefits against the vascular and renal damage caused by diabetes and cardiovascular disease (CVD). Telmisartan demonstrates activity at the peroxisome proliferator-activated receptor delta (PPAR-δ) receptor and activates PPAR-δ receptors in several tissues. Preclinical studies have shown that telmisartan increases revascularization and decreases inflammation in animal models of brain injury. Telmisartan may also decrease apoptotic cell death and oxidative stress associated with impaired mitochondrial function. Telmisartan may also promote the differentiation of oligodendrocyte precursor cells to enhance myelin formation.

Effective amounts of Telmisartan in the daily dose of the compound of the present invention is less than 80 mg, more preferably less than 40 mg, and most preferably less than 20 mg per dose. Effective amounts of Telmisartan in the daily dosage is 1 mg to 80 mg in the compound of the present invention, preferably 2 mg to 40 mg, most preferable 40 mg.

### Duloxetine

Duloxetine, having a chemical structure of C₁₈H₁₉NOS, is a selective serotonin-norepinephrine reuptake inhibitor. It is a medication used to treat major depressive disorder, generalized anxiety disorder, fibromyalgia, neuropathic pain and central sensitization. Serotonin reuptake inhibitors effectively stimulate neurogenesis, which may make a contribution to neurorestoration.

Effective amounts of Duloxetine in the daily dose of the present invention is less than 70 mg, more preferably less than 30 mg, and most preferably less than 25 mg per oral dose or pill. Effective amounts of Duloxetine in the daily dosage is 1 mg to 60 mg in the compound of the present invention, preferably 15 mg to 60 mg.

### DELIVERY METHOD AND EXAMPLES

The neurorehabilitation compound implementing multiple neuroplasticity inducing mechanisms of action may be formed in a variety of delivery methods, however oral delivery or IV delivery is preferred.

One preferred embodiment of the present invention provides a neurorestoration compound implementing multiple neuroplasticity inducing mechanisms of action including pharmacologically effective amounts of Cilostazol and Metformin and Telmisartan. One preferred effective delivery mechanism is in once or twice a day pill format, with once a day being preferred. Effective amounts of Cilostazol in the daily dosage of this embodiment is 1 mg to 200 mg in the compound of the present invention, preferably 10 mg to 100 mg, most preferable 50 mg. The effective amounts of Metformin in the daily dosage of this embodiment are 30 mg to 2000 mg in the compound of the present invention, preferably 40 mg to 850 mg, more preferably 50 mg to 600 mg and 500 mg being an efficient effective amount. Effective amounts of Telmisartan in the daily dosage of this embodiment is 1 mg to 80 mg in the compound of the present invention, preferably 2 mg to 40 mg, most preferable 40 mg.

A second preferred embodiment of the present invention provides a neurorestoration compound implementing multiple neuroplasticity inducing mechanisms of action including pharmacologically effective amounts of Cilostazol and Metformin and Duloxetine. Again, one effective delivery mechanism is in once or twice a day pill format, with once a day being preferred. Effective amounts of Cilostazol in the daily dosage of this embodiment is 1 mg to 200 mg in the compound of the present invention, preferably 10 mg to 100 mg, most preferable 50 mg. The effective amounts of Metformin in the daily dosage of this embodiment are 30 mg to 2000 mg in the compound of the present invention, preferably 40 mg to 850 mg, more preferably 50 mg to 600 mg and 500 mg being an efficient effective amount. Effective amounts of Duloxetine in the daily dosage of this embodiment is 1 mg to 60 mg in the compound of the present invention, preferably 15 mg to 60 mg.

The neurorestoration compounds implementing multiple neuroplasticity inducing mechanisms of action is believed to be particularly well suited for stroke rehabilitation.

While this invention has been particularly shown and described with references to the preferred embodiments thereof, specifically the preferred embodiment of the present invention is directed to stroke rehabilitation protocol implementing robotic assisted rehabilitation in subjects with pharmacologically induced neuroplasticity, it will be understood by those skilled in the art that various changes in form and details may be made therein without departing from the scope of the invention.

Rehabilitation with Compound

Various therapies are often implemented in neuro-restoration such as exercise-based therapy. The neurorehabilitation compound implementing multiple neuroplasticity inducing mechanisms of action may be administered to the patient preferably orally within 4 hours implementing of each therapy session, preferably within 2 hours of each therapy session and generally about one hour before such a therapy session.

It should be apparent from the above description that many variations are possible within the present invention without departing from the spirit and scope thereof, and the scope of the present invention is defined by the appended claims and equivalents thereto.

What is claimed is:

1. A neurorestoration composition implementing multiple neuroplasticity inducing mechanisms of action, wherein the composition is a mixture of at least:
a) a first compound consisting essentially of Cilostazol having the chemical formula C₂₀H₂₇N₅O₂;
b) a second compound consisting essentially of Metformin having the chemical formula C₄H₁₁N₅; and
c) a third compound consisting essentially of Telmisartan having the chemical formula C₃₃H₃₀N₄O₂.

2. The neurorestoration composition according to claim 1 wherein the composition is configured for one of oral or IV delivery.

3. The neurorestoration composition according to claim 2 wherein the composition is configured for oral delivery.

4. The neurorestoration composition according to claim 3 wherein the composition includes amounts of the first compound consisting of Cilostazol in a daily dosage of the composition of 1 mg to 200 mg.

5. The neurorestoration composition according to claim 3 wherein the composition includes amounts of the first compound consisting of Cilostazol in a daily dosage of the composition of 10 mg to 100 mg.

6. The neurorestoration composition according to claim 5 wherein the composition includes amounts of the second compound consisting of Metformin in a daily dosage of the composition of 30 mg to 2000 mg.

7. The neurorestoration composition according to claim 5 wherein the composition includes amounts of the second compound consisting of Metformin in a daily dosage of the composition of 40 mg to 850 mg.

8. The neurorestoration composition according to claim 7 wherein the composition includes amounts of the third compound consisting of Telmisartan in a daily dosage the composition of 1 mg to 80 mg.

9. The neurorestoration composition according to claim 7 wherein the composition includes amounts of the third compound consisting of Telmisartan in a daily dosage of the composition of 2 mg to 40 mg.

10. The neurorestoration composition according to claim 3 wherein the composition includes amounts of the second compound consisting of Metformin in a daily dosage of the composition of 30 mg to 2000 mg.

11. The neurorestoration composition according to claim 3 wherein the composition includes amounts of the second compound consisting of Metformin in a daily dosage of the composition of 40 mg to 850 mg.

12. The neurorestoration composition according to claim 11 wherein the composition includes amounts of the third compound consisting of Telmisartan in a daily dosage of the composition of 1 mg to 80 mg.

13. The neurorestoration composition according to claim 11 wherein the composition includes amounts of the third compound consisting of Telmisartan in a daily dosage of the composition of 2 mg to 40 mg.

14. A neurorestoration composition implementing multiple neuroplasticity inducing mechanisms of action, wherein a daily dosage of the composition is a mixture of at least:
a) 1 mg to 200 mg of Cilostazol;
b) 30 mg to 2000 mg of Metformin; and
c) 1 mg to 80 mg of Telmisartan.

15. The neurorestoration composition according to claim 14 wherein the composition is configured in pill format for oral delivery taken once or twice daily.

16. The neurorestoration composition according to claim 14 wherein the composition is a mixture of at least
a) 10 mg to 100 mg of Cilostazol;
b) 40 mg to 850 mg of Metformin; and
c) 2 mg to 40 mg of Telmisartan.

17. A neurorestoration composition implementing multiple neuroplasticity inducing mechanisms of action comprises a mixture of pharmacologically effective amounts of 1 mg to 200 mg of Cilostazol, 30 mg to 2000 mg of Metformin and at least one of 1 mg to 80 mg of Telmisartan and 1 mg to 60 mg of Duloxetine, wherein the composition is configured for a single daily dosage of one of oral or IV delivery.

18. The neurorestoration composition according to claim 17 wherein the composition is configured for oral delivery.

19. The neurorestoration composition according to claim 17 including pharmacologically effective amounts of Telmisartan.

20. The neurorestoration composition according to claim 19 further including a vasodilator.
